(19) United States
(12) Patent Application Publication
(10) Pub. No.: US 2008/0004509 A1
(43) Pub. Date: Jan. 3, 2008

(54) Assessment of functional status

(75) Inventors: Barbara Sahakian, Cambridge (GB); Andrew Blackwell, Cambridge (GB)

Correspondence Address: MORRISON & FOERSTER LLP, 1650 TYSONS BOULEVARD, SUITE 400, MCLEAN, VA, 22102, US

(73) Assignee: Cambridge University Technical Services Limited, Cambridge (GB)

(21) Appl. No.: 11/808,561

(22) Filed: Jun. 11, 2007

(30) Foreign Application Priority Data
Jun. 9, 2006 (GB) 0611458.1

(51) Int. Cl. A61B 5/00

(52) U.S. Cl. 600/300

(57) ABSTRACT

This invention relates to methods of neurocognitive assessment and, in particular, to methods for assessing the ability of individuals, in particular, memory-impaired individuals, to perform daily activities (i.e. functional status).

### CROSS-REFERENCE TO RELATED APPLICATION

This application claims priority to Great Britain Patent Application No. 0611458.1 filed on Jun. 9, 2006, which is incorporated herein, in its entirety, by reference.

### FIELD OF THE INVENTION

This invention relates to methods of neurocognitive assessment and, in particular, to methods for assessing the ability of individuals, in particular, memory-impaired individuals, to perform daily activities (i.e. functional status).

### BACKGROUND OF THE INVENTION

Visuospatial learning and memory tests, in particular paired associates learning (PAL) tests, are designed to target hippocampal formation and medial temporal lobe dysfunction in the brain. Such tests have been shown to be sensitive to deficits in patients with mild Alzheimer's disease (Sahakian et al 1988, Swainson et al., 2001). Subsequent work has shown that these tests are only sensitive to deficits in Parkinson's disease in advanced cases (Owen et al 1995) and may be impaired following neurosurgical damage to the frontal or temporal lobe, including unilateral hippocampectomy (Owen et al 1993).

Subsequent studies have shown the utility of visuospatial learning and memory tests, such as PAL, as predictive tools in predicting Alzheimer's disease in patients with mild cognitive impairment or questionable dementia presenting to a memory clinic (Swainson et al (2001). PAL tests have been found to be highly predictive of this tendency up to 32 months before formal diagnosis (Blackwell et al 2004). A similar predictive power has been shown in a study of the normal elderly community (De Jager et al 2005).

### SUMMARY OF THE INVENTION

The present inventors have now discovered that performance in visuospatial learning and memory tests, such as PAL, correlates with the current functional status of individuals, in particular individuals with memory impairments, for example as indexed by the CBI and ADL scales. This finding allows the clinician to rapidly assess the functional status of a patient in the clinic. It may also be useful in clinical trials to determine the efficacy of therapeutic strategies in improving the functional status of individuals.

An embodiment of the invention may include a method of assessing the functional status of an individual comprising, providing a visuospatial learning and memory score obtained from an assessment of a visuospatial learning and memory of said individual, and determining from said score a functional status of said individual.

An embodiment of the invention may further comprise assessing the visuospatial learning and memory of said individual to produce the visuospatial learning and memory score.

According to a further embodiment, the functional status score may be determined by relating the visuospatial learning and memory score of the individual with at least one metric.

According to a further embodiment, the at least one metric may be one of: age, gender, IQ, ethnicity, level of education, genetic factors, medical history, current medication and current comorbid disorders.

According to a further embodiment, the visuospatial learning and memory score of the individual may be related to the at least one metric using an algorithm or model to produce a functional status score.

According to a further embodiment, the visuospatial learning and memory may be assessed using a paired associates learning test.

According to a further embodiment, the paired associates learning test may be an automated test configured to be conducted using a computer.

According to a further embodiment, the paired associates learning test may be a CANTAB-PAL test.

An embodiment of the invention may further comprise administering a therapy to the individual.

An embodiment of the invention may further comprise determining the functional status of said individual following said administration.

An embodiment of the invention may include a method of identifying a compound useful for improving a functional status of an individual having a reduced or impaired memory, the method comprising: assessing a visuospatial learning and memory of an individual having a condition characterised by a reduced or impaired memory to produce a first visuospatial learning and memory score for the individual; determining from said first score the functional status of said individual before administration of a test compound; administering the test compound to the individual; assessing the visuospatial learning and memory of said individual to produce a second visuospatial learning and memory score; and determining from said second score the functional status of said individual following said administration.

An embodiment of the invention may include a method of identifying a compound for improving a functional status of an individual having a reduced or impaired memory, the method comprising: providing a population of individuals having reduced or impaired memories; assessing a visuospatial learning and memory of each of the individuals to produce a first visuospatial learning and memory score; determining from said first score the functional status of each individual before an administration of a test compound; administering the test compound to each individual; assessing the visuospatial learning and memory of each individual to produce a second visuospatial learning and memory score; and determining from said second score the functional status of each individual following said administration.

According to a further embodiment, an improvement in the functional status following said administration relative to before the administration may be indicative that the compound is useful for improving the cognitive function of the individual having the reduced or impaired memory.

According to a further embodiment, an improvement in said functional status following said administration relative to at least one control may be indicative that the compound is useful for improving the cognitive function of the individual having the reduced or impaired memory.

According to a further embodiment, the functional status of the individual may be expressed as a functional status score.

According to a further embodiment, the functional status score may be determined by relating at least the first or second visuospatial learning and memory score of the individual to at least one metric.

According to a further embodiment, the at least one metric may be one of: age, gender, IQ, ethnicity, level of education, genetic factors, medical history, current medication and current comorbid disorders.

According to a further embodiment, at least the first or second visuospatial learning and memory score of the individual may be related to the at least one metric using an algorithm or model to produce the functional status score.

According to a further embodiment, the visuospatial learning and memory may be assessed using a paired associates learning test.

According to a further embodiment, the paired associates learning test may be configured to be conducted using a computer.

According to a further embodiment, the paired associates learning test may be a CANTAB-PAL test.

According to a further embodiment, a computer-readable medium may be configured to perform any of the disclosed methods.

An embodiment of the invention may include a computer system comprising a processor configured to perform a visuospatial learning and memory test on a subject, record a plurality of responses by the subject to the test, calculate a visuospatial learning and memory score for the subject based on the plurality of responses, and determine a functional status of the subject based on the visuospatial learning and memory score.

According to a further embodiment, the processor may be further configured to produce a functional status score.

According to a further embodiment, the functional status score may be to be displayed on a monitor.

According to a further embodiment, the functional status score may be to be recorded and/or stored in a data storage device.

An embodiment of the invention may include a method of assessing a functional status, comprising assessing a plurality of individuals to determine a plurality of metrics for each of the individuals, wherein the metrics comprise physical characteristics of the individuals; performing a visuospatial learning and memory test on each of the plurality of individuals to determine a visuospatial learning and memory score for each of the individuals; and determining a functional status for each of the individuals based on the plurality of metrics and the visuospatial learning and memory score of each individual.

According to a further embodiment, the plurality of metrics may further comprise mental characteristics of the individuals.

According to a further embodiment, the plurality of metrics may further comprise health histories of the individuals.

An embodiment of the invention may further comprise identifying a subset of the individuals based on the functional status of the individuals; administering a test compound to the subset of individuals; performing a second visuospatial learning and memory test on each of the plurality of individuals in the subset to determine a second visuospatial learning and memory score for each of the individuals in the subset; determining a post administration functional status for each of the individuals in the subset based on the plurality of metrics and the second visuospatial learning and memory score of each individual; and determining the effect of the test compound on each of the individuals in the subset based on a difference between the individual's functional status prior to the administration of the test compound and the individual's post administration functional status.

An embodiment of the invention may further comprise determining the effect of the test compound based on the effect of the test compound on each of the individuals.

According to a further embodiment, the method may be used to monitor the effects of the test compound.

An embodiment of the invention may further comprise basing prescription decisions on the monitored effects of the test compound.

An embodiment of the invention may include a method of assessing a functional status, comprising determining a first functional status of a subject based on a plurality of metrics related to the subject and results of a visuospatial learning and memory test, administering a test compound to the subject; determining a second functional status for the subject based on the plurality of metrics and a second visuospatial learning and memory test performed following the administration of the test compound; and determining the effect of the test compound on the subject based on a difference between the first functional status and the second functional status.

An embodiment of the invention may further comprise determining at least on additional functional status of the subject following at least one additional administration of the test compound.

An embodiment of the invention may further comprise monitoring the subject's functional status.

An embodiment of the invention may further comprise determining prescription parameters based at least in part on the monitoring of the subject's functional status.

### BRIEF DESCRIPTION OF THE DRAWINGS

Certain aspects and embodiments of the invention will now be illustrated by way of example and with reference to the figures and tables described below.

FIG. 1 shows the correlation between CANTAB PAL performance and CBI memory score for 67 subjects tested.

FIG. 2 shows the correlation between CANTAB PAL performance and CBI total score for 67 subjects tested.

### DETAILED DESCRIPTION OF THE INVENTION

One aspect of the invention provides a method of assessing the functional status of an individual comprising: providing a visuospatial learning and memory score obtained from an assessment of the visuospatial learning and memory of said individual, and; determining from said score the functional status of said individual.

A method may comprise assessing the visuospatial learning and memory of said individual to produce the visuospatial learning and memory score.

The visuospatial learning and memory score may be indicative of the functional status of the individual. The score may be compared with standard or control scores, for example from individuals of known functional status. A low visuospatial learning and memory score relative to controls may, for example, be indicative of low functional status.

Functional status may be expressed as a functional status score. The functional status score may be a numerical value, class, classification, category, band or grade which indicates or reflects the functional status of said individual.

The functional status score may be determined from the visuospatial learning and memory score, for example using coefficients derived from multiple regression analyses of the association between functional status and visuospatial learning and memory score.

The functional status score may be determined by relating the visuospatial learning and memory score of the individual with predetermined values or standard or control scores for individuals or populations of individuals of known functional status (for example, as assessed by known techniques). In some embodiments, this may be performed by direct comparison. For example, the functional status score may be expressed as a ‘pass’ if the visuospatial learning and memory score is above a predetermined threshold value and ‘fail’ if below, or it may be expressed as a proportion of the population (e.g. a percentile) having a greater or lower visuospatial learning and memory score than the assessed individual.

In other embodiments, the visuospatial learning and memory score of the individual may be related to predetermined values or controls using an algorithm or model to produce a functional status score. The algorithm or model may include additional parameters, such as age, gender, IQ, ethnicity, level of education, genetic factors, medical history (e.g. head injury), current medication and current comorbid disorders (such as depression). A suitable algorithm or model may be produced from the visuospatial learning and memory scores of a sample or population of individuals who are also assessed for functional status by conventional means, such as multiple regression analyses. Examples of conventional instruments for evaluating functional status include Cambridge Behavioural Inventory (CBI) (Bozeat et al 2001), Activities of Daily Living (ADL) (Katz 1963), and ‘SF-36’ (Mchorney et al 1993).

A method of producing an algorithm or model for determining the functional status of an individual may comprise; assessing the visuospatial learning and memory ability of a sample or population of individuals, to produce visuospatial learning and memory scores for each member of said sample or population; assessing the functional status of the members of the sample or population, and; relating the visuospatial learning and memory scores of each of said members with the assessed functional status to produce an algorithm which relates said test scores to the functional status of the individual.

The visuospatial learning and memory scores of each of the members of the sample or population may be related with the assessed functional status of the member, optionally in combination with one or more of the age, gender, IQ, ethnicity, level of education, genetic factors, medical history (e.g. head injury), current medication and current comorbid disorders (such as depression) of the member of the sample or population.

The assessment of the functional status of the members of the sample or population may produce a functional status score for each of the members. The visuospatial learning and memory scores may be related to the functional status scores of the members of the sample or population, for example using multiple regression analyses, to produce the algorithm.

An individual may then be assessed for functional status by producing a visuospatial learning and memory score for the individual as described above; and, applying the algorithm to the score of the individual to produce a functional status score which is indicative of the functional status of said individual.

The methods described herein allow the functional status of the individual at the time of the assessment to be determined (i.e. the current functional status of the individual). Functional status is the ability to carry out activities of daily living, including self-care, self-maintenance, and physical activities. An individual with high functional status may be capable of carrying out daily living activities without assistance. An individual with low functional status may be incapable of carrying out daily living activities without assistance. In particular, functional status may include the status or ability of an individual with regard to orientation and attention, everyday skills, ability to self-care, beliefs and/or motivation. An assessment of functional status as described herein may be useful in determining the level of care and assistance that is required by the individual to carry out daily living activities.

An assessment of functional status may also be useful in determining the ability of an individual to perform occupational tasks (i.e. the occupational status of the individual). For example, an individual with high functional status may be capable of carrying out the tasks required for a particular occupation. An individual with low functional status may be unable to carry out such tasks without additional support or assistance.

The individual may have a normal or unimpaired memory or may have a reduced or impaired memory. A reduced or impaired memory includes any degree of memory loss or reduced performance in memory tests relative to controls. Controls may be age-adjusted to evaluate memory relative to the normal population of the same age or non-age adjusted, to evaluate memory relative to the normal population of young adults.

In some embodiments, the individual may have a condition which is characterised by memory impairment, such as questionable dementia, Alzheimer's disease, schizophrenia, Age-related Cognitive Decline, age associated memory impairment, mild cognitive decline, head trauma, stroke, major unipolar depression, mild cognitive impairment, cognitive impairment with no dementia, Parkinson's disease, bipolar depression, Huntington's disease, normal pressure hydrocephalus, multiple sclerosis, other forms of dementia and neurodegenerative disease and first episode psychosis. Neuropsychiatric diagnostic criteria for these disorders are set out, for example in the Diagnostic and Statistical Manual of Mental Disorders (text revision), American Psychiatric Association (2000) American Psychiatric Publishing Inc (DSM-IV-TR).

In some embodiments, the methods described herein may be accompanied by further assessment of the condition. For example, the individual may be assessed for one or more additional neuropsychiatric diagnostic criteria for the condition, for example as set out in DSM-IV. In other embodiments, the individual may not be assessed for one or more additional neuropsychiatric diagnostic criteria for the condition.

In some embodiments, the visuospatial memory and learning ability of the individual may be assessed at a single time point to determine functional status at that time point. In other embodiments, the visuospatial memory and learning of the individual may be determined at two or more time points. Suitable time points may, for example, be 1, 2, 3 or 4 or more years apart. This allows the changes in the functional status of the individual to be identified and monitored.

Visuospatial memory and learning ability is preferably assessed using a paired associates learning test. Various forms of paired associates learning test or related tests are known in the art including Wechsler verbal paired associates learning test and Busche Selective reminding test. Preferably, the test is an automated test conducted by a computer system, for example by means of a touch sensitive monitor. In preferred embodiments, the Cambridge Neuropsychological Test Automated Battery (CANTAB) visuospatial paired associates learning (PAL) test may be used (Sahakian et al. (1988) Brain 111: 695-718; Cambridge Cognition Ltd, Cambridge UK).

CANTAB PAL involves the sequential display of 1, 2, 3, 6 or 8 patterns in boxes on a display. Each pattern is then presented in the centre of the display and the subject is required to touch the box in which the pattern was previously seen. If all the responses are correct, the test moves on to the next stage; an incorrect response results in all the patterns being redisplayed in their original locations, followed by another recall phase. The task terminates after 10 presentations and recall phases if all patterns have not been placed correctly. The test may be scored in a variety of ways, including for example number of stages passed. Preferably, the test is scored by the total number of trials during the test. This may be adjusted for individuals who do not complete the test. Other PAL tests may be employed in a similar way to determine functional status.

Visuospatial memory and learning ability may also be assessed using memory or recognition memory tests with abstract stimuli or non-abstract stimuli morphed to appear abstract. A number of suitable tests are known in the art (see, for example, Lezak et al chapter 11 p 450 ‘tests of visual memory’).

On the basis of the assessed functional status of the individual, a therapy, for example an anti-dementia or cognitive enhancing therapy, may be administered to the individual. For example, an individual having a functional status below a predetermined threshold value may be treated with an anti-dementia therapy. The functional status of the individual may be assessed after administration or treatment with the therapy to determine the effect of the therapy on the functional status of the individual.

Anti-dementia therapy may include, for example, administration of cholinesterase inhibitors, such as Aricept™, Exelon™ and Reminyl™, statins, NMDA antagonists, amyloid therapies, anti-inflammatories, oestrogen, anti-oxidants, ampakines, nootropics, secretase inhibitors, nicotinic treatments, anti-amyloid vaccines, vitamin therapies or other glutamate receptor modulators, such as Ebixa™.

Administration of an anti-dementia therapy may be effected in one dose, continuously or intermittently (e.g. in divided doses at appropriate intervals) throughout a course of treatment as determined by the treating physician. Methods of determining the most effective means and dosage of administration are well-known to those of skill in the art and will vary with the formulation used for therapy, and the subject being treated. Single or multiple administrations can be carried out with the dose level and pattern being selected by the treating physician.

Other aspects of the invention relate to methods for determining or assessing the efficacy of treatments for improving cognitive function of individuals with reduced or impaired memory.

A method of identifying a compound useful for improving the cognitive function of an individual having a reduced or impaired memory or a compound useful for improving the functional status of an individual having a reduced or impaired memory, may comprise, assessing the visuospatial learning and memory of said individual to produce a first visuospatial learning and memory score for the individual, determining from said first score the functional status of said individual before administration of a test compound, administering the test compound to the individual; assessing the visuospatial learning and memory of said individual to produce a second visuospatial learning and memory score; and, determining from said second score the functional status of said individual following said administration.

An individual having a reduced or impaired memory may include an individual having a condition associated with impaired memory.

The effect of the test compound on the individual may be determined from the functional status of said individual before and after said administration. An improvement in the functional status of the individual after the administration of the test compound relative to before administration of the test compound, or a stabilisation or reduced rate of decline of functional status, may be indicative that the test compound is useful in improving the cognitive function of the individual or improving the functional status of the individual having reduced or impaired memory. An improvement in the functional status following said administration relative to controls (e.g. individuals treated with placebo) may also be indicative that the compound is useful in improving the cognitive function of the individual or improving the functional status of the individual having reduced or impaired memory.

Any pharmaceutical agent with a suitable safety profile for administration to a human may be employed as a test compound. A suitable compound may be a known compound for use in treating conditions characterise by memory impairment, for example an cholinesterase inhibitor, statin, NMDA antagonist, amyloid therapy, anti-inflammatory, oestrogen, nicotinic treatment, anti-amyloid vaccine, anti-oxidant, ampakine, nootropic, secretase inhibitor, vitamin therapy or other glutamate receptor modulator or a compound not previously known for use in treating such conditions.

Protocols and approaches for performing such trials, including the provision of suitable controls, are well known to the skilled person in the field.

Appropriate means and dosage of administration of the test compound may be determined by the treating physician and will vary with the compound, formulation and the subject being treated.

In some embodiments, the effect of a test compound on a population of individuals may be determined in order to identify a compound useful for improving the cognitive function of an individual having a reduced or impaired memory or a compound useful for improving the functional status of an individual having a reduced or impaired memory. A method of identifying such a compound may comprise, providing a population of individuals having reduced or impaired memory, assessing the visuospatial learning and memory of each of the members of the population to produce a first visuospatial learning and memory score, determining from said first score the functional status of each member of the population before administration of a test compound, administering the test compound to the population; assessing the visuospatial learning and memory of each member of the population to produce a second visuospatial learning and memory score, and; determining from said scores the functional status of each member of the population following said administration.

The effect of the test compound on the population may be determined from the functional status of each member of said population before and after said administration. An improvement in the functional status of the population after the administration of the test compound relative to before administration of the test compound is indicative that the test compound is useful in treating the condition or improving the functional status of an individual having the condition.

Visuospatial learning and memory ability may be assessed using a paired associates learning test. Suitable paired associates learning tests such as the CANTAB-PAL test (Cambridge Neuropsychological Test Automated Battery visuospatial paired associates learning), are described in more detail above.

In preferred embodiments of the methods described herein, the visuospatial learning and memory is assessed and functional status determined using automated means, for example a computer system which performs the visuospatial learning and memory assessment of the individual, determines the visuospatial learning and memory score for the assessed individual and determines the functional status of the assessed individual from the visuospatial learning and memory score.

Further aspects of the invention provide: (i) computer-readable code for performing a method described herein, (ii) a computer program product carrying such computer-readable code, and (iii) a computer system configured to perform a method described herein.

A computer readable code may comprise instructions to cause a computer system comprising a processor and a display to carry out the following steps when loaded into said processor; perform a visuospatial learning and memory test on a subject and record the responses of the subject to the test, calculate a visuospatial learning and memory score for the subject from the responses of the subject to the test, and, determine the functional status of the subject from the visuospatial learning and memory score.

The term “computer program product” includes any computer readable medium or media which can be read and accessed directly by a computer. Typical media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

A typical computer system of the present invention comprises a central processing unit (CPU), input device, output device and data storage device (such as RAM or ROM). A monitor or other image display is preferably provided.

The input device may comprise a mouse, keyboard, touch sensitive monitor or other graphic interface device which allows the subject to undertake the visuospatial learning and memory test.

For example, a computer system may comprise a processor adapted to perform a method of the invention. For example the processor may be adapted to: perform a visuospatial learning and memory test on a subject and record the responses of the subject to the test, calculate a visuospatial learning and memory score for the subject from the responses of the subject to the test, and, determine the functional status of the subject from the visuospatial learning and memory score.

The visuospatial learning and memory test may be performed by displaying test images on the display and recording the responses of the subject to the displayed test images which are entered via the input device.

The functional status is determined in the form of a functional status score, which may be a numerical value, classification, band or grade which is indicative of the functional status of the subject. The functional status score may be displayed on a monitor or recorded and/or stored in the data storage device for subsequent access by a clinician.

The data storage device may store the responses of the subject at each stage of the test and/or record the visuospatial learning and memory score and functional status scores. The data storage device may be adapted for storing assessment test results from a number of different individuals. Statistics and data derived from these test results, for example a functional status score for an individual, may be stored on another or the same data storage device, and/or may be sent to an output device or displayed on a monitor.

Another aspect of the invention provides a test device for assessing an individual for functional status comprising a display, a graphic interface and a processor adapted for use in a method described herein.

Various further aspects and embodiments of the present invention will be apparent to those skilled in the art in view of the present disclosure. All documents mentioned in this specification are incorporated herein by reference in their entirety.

### EXAMPLES

Table 1 shows the association between CANTAB PAL performance and functional status, as assessed by CBI and ADL.

Two subject groups completed the PAL and CBI assessments: Probable AD (n=37); questionable dementia (n=29). At baseline, consecutive referrals to the Memory Clinic at Addenbrooke's Hospital, Cambridge, were approached and screened for suitability for inclusion into the study; where necessary, other patients were recruited from the Psychiatry Department of West Suffolk Hospital and from the Mental Health Services of Addenbrooke's NHS Trust. All subjects were aged between 50 and 80 years at time of recruitment and were screened for the following exclusion criteria: extrapyramidal signs or hallucinations; vascular dementias; current cancer treatment (radiotherapy or chemotherapy); uncontrolled diabetes; serious head injury requiring surgical intervention. Patients having suffered cerebrovascular events (e.g. TIA or stroke) or epilepsy were excluded at the discretion of a senior neurologist. Demographic and clinical data for the sample are shown in Table 1.

In the clinic, many patients presenting with deficits in memory function do not fulfil a neurological or psychiatric diagnosis. These patients—who therefore did not meet the diagnostic criteria for probable AD (NINCDS-ADRDA; [McKhann, 1984] or Major Unipolar Depression (DSM-IV; [American Psychiatric Association, 1994])—were allocated to the questionable dementia (QD) group. Included within this group were, therefore, individuals with subjective complaints of memory loss yet showing normal performance on objective tests as well as those with substantial objectively-defined deficits restricted to memory impairment.

All AD patients met the NINCDS-ADRDA [McKhann, 1984] criteria for probable AD. AD patients scoring below 17 on the MMSE at baseline were not included because of the expected difficulty in administering computerised tests to such patients after 2 years.

For the PAL test the performance measure used was ‘total trials adjusted’. This measure represents the total number of presentations required (maximum score=10 presentations per trial) to locate all the patterns correctly in all trials. When using this measure it is important to analyse the data with reference to the PAL Sets completed score. This is because subjects who fail to complete the test will have had fewer Total trials simply because they had less opportunity to make errors than subjects who completed the test. One possible way of dealing with this is to add the maximum score of 10 trials for each stage not attempted due to an earlier failure and this is what this metric shows.

For the Cambridge Behavioural Inventory (CBI) (Bozeat et al 2001), which is a carer rated scale of the patients' behavioural, emotional and cognitive day-to-day activities, the measures used were subscale scores reflecting defined categories of functional behaviour together with a ‘total score’.

For the Activities of Daily Living (ADL), which is a carer rating scale of patients cognitive and behavioural functioning, each of the six subscales were scored independently.

The data used to establish this relationship were collected at the final visit of a prospective longitudinal study 32 months after baseline assessment. See Swainson et al. (2001).

The degree of association between performance on the CANTAB PAL test and functional status was examined using Pearson's correlation coefficient. Table 1 details the associations between PAL performance and total functional status score and each subtest score.

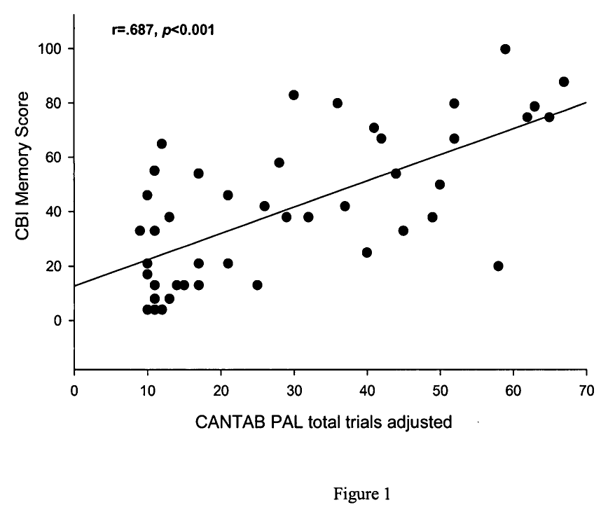

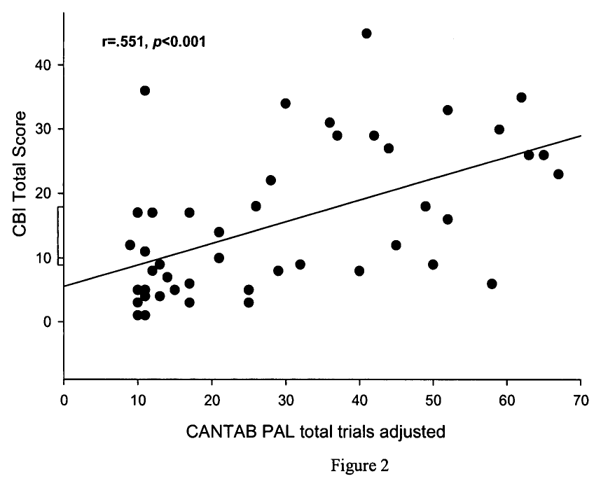

As can be seen in FIGS. 1 and 2, a statistically significant association was identified between CANTAB PAL performance and CBI memory score and CBI total score. Other significant associations with PAL performance include: Orientation and Attention, Everyday skills, Self-care, Beliefs and Motivation (see table 1).

These associations found with CBI are supported by related or similar associations between CANTAB PAL and ADL (see table 1).

The data set out above shows that a statistically significant association exists between CANTAB PAL performance and functional status as indexed by the CBI and ADL scales. This association provides evidence of the ecological validity of CANTAB PAL. For the clinician, this association means that inferences can be drawn about functional status of patients on the basis of PAL performance, a brief objective test which can be performed in the clinic. For pharmaceutical companies and regulatory bodies this means that an effect of drug on CANTAB PAL performance may indicate that the drug is likely to impact upon functional status in the home. This is a key factor in adjudging the value of a drug or other therapeutic strategy.

### REFERENCES

Sahakian B J et al Brain 1988; 111 (Pt 3):695-718.
Swainson R et al Dement Geriatr Cogn Disord 2001; 12:265-80.
De Jager Cet al Am J Geriatr Psychiatry. August 2005;13(8):735-40.
Owen A M, et al Neuropsychologia. January 1995;33(1):1-24.
Owen A M, et al Neuropsychologia. July 1993;31 (7):627-44.
Blackwell A D, et al Dement Geriatr Cogn Disord. 2004;17(1-2):42-8. Epub Oct. 13, 2003.
Bozeat S, et al J Neurol Neurosurg Psychiatry. August 2000;69(2):178-86.
M. D. Lezak, D. B. Howieson, and D. W. Loring (Eds.), Neuropsychological Assessment, 4th ed. New York: Oxford University Press.
Katz Set al. JAMA 1963;185:914-919.
McHorney C A et al The MOS 36-Item Short-Form Health Survey (SF-36), II: psychometric and clinical tests of validity in measuring physical and mental health constructs. Med Care 31 (3): 247-263, 1993.
Diagnostic and Statistical Manual of Mental Disorders (4th edition) (DSM-IV). APA, Washington, D.C., (1994).
McKhann G, et al Neurology 1984; 34:939-44.
Buschke H. Spreen O & Strauss E (1991). A Compendium of neuropsychological tests: Administration, norms, and commentary. NY: Oxford University Press. [list of 16 complete instruments]. Pg. 125-138
Braak H, Braak E: Acta Neuropathol (Berl) 1991; 82:239-59.
Owen A M et al Journal of Cognitive Neuroscience 1996; 8:588-602.
Declaration of Helsinki. Recommendations guiding physicians in biomedical research involving human subjects (1996).
Folstein M F et al J Psychiatr Res 1975; 12:189-98.
Wechsler D: Wechsler Memory Scale—Revised. Psychological Corporation, San Antonio, Calif. (1987).

de Jager C A, et al Psychol Med 2002; 32:483-91.

TABLE 1

| | Correlation | |
|---|---|---|
| | with PAL | |
| | (total trials | |
| | adjusted) | P |
| CBI measure | | |
| Total Score | .551 | <0.0001 |
| Memory | .687 | <0.0001 |
| Orientation and attention | .652 | <0.0001 |
| Everyday skills | .583 | <0.0001 |
| Self care | .435 | 0.003 |
| Mood | .139 | 0.356 n.s. |
| Beliefs | .446 | 0.002 |
| Challenging behaviour | −.035 | 0.819 n.s |
| Disinhibition | −.011 | 0.945 n.s |
| Eating habits | .185 | 0.225 n.s |
| Sleep | −.015 | 0.920 n.s |
| Stereotypic behaviours | .194 | 0.201 n.s |
| Motivation | .431 | 0.003 |
| Insight | −.055 | 0.733 n.s |
| ADLs | | |
| Memory 1-5 | .581 | <0.0001 |
| Orientation 1-4 | .573 | <0.0001 |
| Judgement etc 1-5 | .603 | <0.0001 |
| Job, shopping etc 1-4 | .604 | <0.0001 |
| Home 1-5 | .664 | <0.0001 |
| Personal care 1-4 | .459 | 0.002 |

1. A method of assessing the functional status of an individual comprising:
providing a visuospatial learning and memory score obtained from an assessment of a visuospatial learning and memory of said individual, and
determining from said score a functional status of said individual.

2. The method according to claim 1, further comprising assessing the visuospatial learning and memory of said individual to produce the visuospatial learning and memory score.

3. The method according to claim 1, wherein the individual has a reduced or impaired memory.

4. The method according to claim 1, wherein the functional status of the individual is expressed as a functional status score.

5. The method according to claim 1, wherein the functional status score is determined by relating the visuospatial learning and memory score of the individual with at least one metric.

6. The method according to claim 1, wherein the at least one metric is one of: age, gender, IQ, ethnicity, level of education, genetic factors, medical history, current medication and current comorbid disorders.

7. The method according to claim 6, wherein the visuospatial learning and memory score of the individual is related to the at least one metric using an algorithm or model to produce a functional status score.

8. The method according to claim 2, wherein the visuospatial learning and memory is assessed using a paired associates learning test.

9. The method according to claim 8, wherein the paired associates learning test is an automated test configured to be conducted using a computer.

10. The method according to claim 9, wherein the paired associates learning test is a CANTAB-PAL test.

11. The method according to claim 2, further comprising administering a therapy to the individual.

12. The method according to claim 11, further comprising determining the functional status of said individual following said administration.

13. A method of identifying a compound useful for improving a functional status of an individual having a reduced or impaired memory, the method comprising:
assessing a visuospatial learning and memory of an individual having a condition characterised by a reduced or impaired memory to produce a first visuospatial learning and memory score for the individual;
determining from said first score the functional status of said individual before administration of a test compound;
administering the test compound to the individual;
assessing the visuospatial learning and memory of said individual to produce a second visuospatial learning and memory score; and
determining from said second score the functional status of said individual following said administration.

14. A method of identifying a compound for improving a functional status of an individual having a reduced or impaired memory, the method comprising:
providing a population of individuals having reduced or impaired memories;
assessing a visuospatial learning and memory of each of the individuals to produce a first visuospatial learning and memory score;
determining from said first score the functional status of each individual before an administration of a test compound;
administering the test compound to each individual;
assessing the visuospatial learning and memory of each individual to produce a second visuospatial learning and memory score; and
determining from said second score the functional status of each individual following said administration.

15. The method according to claim 13, wherein an improvement in the functional status following said administration relative to before the administration is indicative that the compound is useful for improving the cognitive function of the individual having the reduced or impaired memory.

16. The method according to claim 13, wherein an improvement in said functional status following said administration relative to at least one control is indicative that the compound is useful for improving the cognitive function of the individual having the reduced or impaired memory.

17. The method according to claim 13, wherein the functional status of the individual is expressed as a functional status score.

18. The method according to claim 17, wherein the functional status score is determined by relating at least the first or second visuospatial learning and memory score of the individual to at least one metric.

19. The method according to claim 18, wherein the at least one metric is one of: age, gender, IQ, ethnicity, level of education, genetic factors, medical history, current medication and current comorbid disorders.

20. The method according to claim 18, wherein at least the first or second visuospatial learning and memory score of the individual is related to the at least one metric using an algorithm or model to produce the functional status score.

21. The method according to claim 13, wherein the visuospatial learning and memory is assessed using a paired associates learning test.

22. The method according to claim 21, wherein the paired associates learning test is configured to be conducted using a computer.

23. The method according to claim 21, wherein the paired associates learning test is a CANTAB-PAL test.

24. A computer-readable medium configured to perform the method of claim 1.

25. A computer-readable medium configured to perform the method of claim 13.

26. A computer-readable medium configured to perform the method of claim 14.

27. A computer system configured to perform the method according to claim 1.

28. A computer system configured to perform the method according to claim 13.

29. A computer system configured to perform the method according to claim 14.

30. A computer system comprising:
a processor configured to:
perform a visuospatial learning and memory test on a subject,
record a plurality of responses by the subject to the test,
calculate a visuospatial learning and memory score for the subject based on the plurality of responses, and
determine a functional status of the subject based on the visuospatial learning and memory score.

31. The computer system according to claim 30, wherein the processor is further configured to produce a functional status score.

32. The computer system according to claim 31, wherein the functional status score is to be displayed on a monitor.

33. The computer system according to claim 32, wherein the functional status score is to be recorded and/or stored in a data storage device.

34. A method for assessing a functional status, comprising:
assessing a plurality of individuals to determine a plurality of metrics for each of the individuals, wherein the metrics comprise physical characteristics of the individuals;
performing a visuospatial learning and memory test on each of the plurality of individuals to determine a visuospatial learning and memory score for each of the individuals; and
determining a functional status for each of the individuals based on the plurality of metrics and the visuospatial learning and memory score of each individual.

35. The method of claim 34, wherein the plurality of metrics further comprise mental characteristics of the individuals.

36. The method of claim 34, wherein the plurality of metrics further comprise health histories of the individuals.

37. The method of claim 34, further comprising:
identifying a subset of the individuals based on the functional status of the individuals;
administering a test compound to the subset of individuals;
performing a second visuospatial learning and memory test on each of the plurality of individuals in the subset to determine a second visuospatial learning and memory score for each of the individuals in the subset;
determining a post administration functional status for each of the individuals in the subset based on the plurality of metrics and the second visuospatial learning and memory score of each individual; and
determining the effect of the test compound on each of the individuals in the subset based on a difference between the individual's functional status prior to the administration of the test compound and the individual's post administration functional status.

38. The method of claim 37, further comprising determining the effect of the test compound based on the effect of the test compound on each of the individuals.

39. The method of claim 37, wherein the method is used to monitor the effects of the test compound.

40. The method of claim 39, further comprising basing prescription decisions on the monitored effects of the test compound.

41. A method for assessing a functional status, comprising:
determining a first functional status of a subject based on a plurality of metrics related to the subject and results of a visuospatial learning and memory test,
administering a test compound to the subject;
determining a second functional status for the subject based on the plurality of metrics and a second visuospatial learning and memory test performed following the administration of the test compound; and
determining the effect of the test compound on the subject based on a difference between the first functional status and the second functional status.

42. The method of claim 41, further comprising determining at least on additional functional status of the subject following at least one additional administration of the test compound.

43. The method of claim 42, further comprising monitoring the subject's functional status.

44. The method of claim 43, further comprising determining prescription parameters based at least in part on the monitoring of the subject's functional status.
